SEQUENCE LISTING (2 sequences)

SEQ ID NO: 1 - RNA, 19 nt, synthetic construct
  source 1..19: mol_type = other RNA
ggcggctctcaggggctgt

SEQ ID NO: 2 - DNA, 5519 bp, synthetic construct
  source 1..5519: mol_type = other DNA
tgacaaccccaaggtgtgagaccctgagggcgcaatggtaacctgaagatagggagagaggggaggactcgcgctctccagcggggcacaccaaccacggccctcccgtggcctcccacgaccactcagccacccctgcacactctggcccacccttgtgccccggcccctctaggcccgcctcctcccgcccctgccccgcccctttggctctgcccctgttgacaccgccccagggcacgcaggccccaccaggcccgctccggagactttcacgtccagggccagccagcagccccgggctgcgcccccgcgcccacccccaccagggcccgccctaaccccgccgccccgcagacctacgtgcaggacatcctgaggacggagctggctgcggaggtgcaccgcgtgctgtgcctcgagcggggccacatgtttgtctgcggcgatgttaccatggcaaccaacgtcctgcagaccgtgcagcgcatcctggcgacggagggcgacatggagctggacgaggccggcgacgtcatcggcgtgctgcgggtgcggaggggcgggccgggcctgagcgtgcggggttcctgctaaggtctccgagtcgggttctgatccactgtgctcttttccgacaggatcagcaacgctaccacgaagacattttcgggctcacgctgcgcacccaggaggtgacaagccgcatacgcacccagagcttttccttgcaggagcgtcagttgcggggcgcagtgccctgggcgttcgaccctcccggctcagacaccaacagccccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctacccggggggtaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaggcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgagatcttgaagttcctatactttctagagaataggaacttcacgaagttatattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccgtcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcagcacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacgatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgcccccgattcgcagcgcatcgccttctatcgccttcttgacgagttcttctgagcgggactctggggttcgaataaagaccgaccaagcgacgtctgagagctccctggcgaattcggtaccaataaaagagctttattttcatgatctgtgtgttggtttttgtgtgcggcgcgataacttcgtatagctgaagttcctatactttctagagaataggaacttcgctagcctttcccttccagttccgggagagcggctgcccgactcaggtccgcccgaccaggatcagccccgctcctcccctcttgaggtggtgccttctcacatctgtccagaggctgcaaggattcagcattattcctccaggaaggagcaaaacgcctcttttccctctctaggcctgttgcctcgggcctgggtccgccttaatctggaaggcccctcccagcagcggtaccccagggcctactgccacccgcttcctgtttcttagtcgaatgttagattcctcttgcctctctcaggagtatcttacctgtaaagtctaatctctaaatcaagtatttattattgaagatttaccataagggactgtgccagatgttaggagaactactaaagtgcctaccccagctcatgtggattacagttttttttttttgtttttttttttttgaaacggagtctccctctgccgcccgggctggagtgcagtggcgtgatctcagctcactgcaacctccaccccacaagttcaagtgattctcctgcctcagcctcccaagtagttgggattacaggtgcctgccaccgcgcccggctaggttttgtatttttagtaaagacggggtttcaccatcttggccaggctggtcttgaactcctgacctcgtgatccaaccgcctcagcctcccaaagtgctgggattacaggtgtgagctactgcacccggcgtggattacaattataaaatgacaagatttctgttttaacctgtgcagttgtgggtatgtggtggggaaaggggtcattcttttgacagagt

(19) United States
(12) Patent Application Publication
(10) Pub. No.: US 2024/0319173 A1
(43) Pub. Date: Sep. 26, 2024

(54) REAGENTS AND METHODS FOR PROMOTING ARTERIAL ENDOTHELIUM DIFFERENTIATION AND NITRIC OXIDE PRODUCTION

(71) Applicant: Wisconsin Alumni Research Foundation, Madison, WI (US)

(72) Inventors: James Thomson, Madison, WI (US); Jue Zhang, Madison, WI (US)

(21) Appl. No.: 18/613,041

(22) Filed: Mar. 21, 2024

Related U.S. Application Data
(60) Provisional application No. 63/453,504, filed on Mar. 21, 2023.

(51) Int. Cl. G01N 33/50; A61K 31/137; A61K 31/202; A61K 31/365; A61K 31/40; A61K 31/4166; A61K 31/47; A61K 31/51; A61K 31/519; A61K 31/55; A61K 31/56; A61K 31/63; A61K 31/685; C12N 5/071; C12P 3/00

(52) U.S. Cl. CPC G01N 33/5073; A61K 31/137; A61K 31/202; A61K 31/365; A61K 31/40; A61K 31/4166; A61K 31/47; A61K 31/51; A61K 31/519; A61K 31/55; A61K 31/56; A61K 31/63; A61K 31/685; C12N 5/069; C12P 3/00; C12N 2506/45; C12N 2510/00

(57) ABSTRACT

The present disclosure provides reagents and methods for identifying compounds that promote arterial endothelial cell differentiation and nitric oxide production therefrom. Pharmaceutical compositions comprising compounds identified thereby are provided as are therapeutic methods using these pharmaceutical compositions for treating neural and cardiovascular diseases and disorders associated with deficient or disrupted nitric oxide production in arterial endothelial cells.

### CROSS-REFERENCE TO RELATED APPLICATIONS

This application claims priority to U.S. provisional application No. 63/453,504, filed Mar. 21, 2023, the disclosure of which is expressly incorporated by reference herein.

### STATEMENT REGARDING FEDERALLY SPONSORED RESEARCH

This invention was made with government support under HL134655 awarded by the National Institutes of Health. The government has certain rights in this invention.

### INCORPORATION BY REFERENCE OF SEQUENCE LISTING PROVIDED ELECTRONICALLY

This application contains a Sequence Listing submitted as an electronic text file named “23-0185-US_SequenceListing_ST26.xml” having a size of 8,359 bytes, and created on Feb. 27, 2024. The information contained in this electronic file is hereby incorporated by reference in its entirety.

### BACKGROUND OF THE DISCLOSURE

Nitric oxide (NO) plays key roles in cardiovascular and nervous diseases. NO regulates vascular smooth muscle cell (VSMC) relaxation, inhibits VSMC proliferation, mediates angiogenesis, and suppresses thrombosis and atherosclerosis. NO also regulates brown fat metabolism and prevents and limits ischemia-reperfusion injuries. NO mediates neurodegeneration in numerous diseases of the nervous system, including Parkinson's disease, Alzheimer's disease, amyotrophic lateral sclerosis, Huntington's disease, and ischemic brain injury (stroke). NOS3 (eNOS) is principally expressed in endothelial cells, but also in cardiomyocytes, platelets, and probably erythrocytes.

Nitric oxide (NO) also plays a significant role in regulating the function of hematopoietic stem cells (HSCs) (Lapidot et al., 2020, Blood Sci. 2(2): 66-67). It has been shown to mediate HSC migration, homing, and repopulation. NO can upregulate expression and function of surface CXCR4, a receptor involved in HSC migration, and enhance HSC motility both in vitro and in vivo. Additionally, NO has been implicated in proliferation and differentiation of HSCs, acting as a switch to balance HSC retention in the niche and their mobilization (Hummar et al., 2020, Front Cell Dev Bio. 8: 610369). Furthermore, NO has contrasting age-dependent effects on HSC functionality, with its role being important in both embryonic and adult hematopoiesis (Jalnapurkar et al., 2016, Stem Cell Res Ther. 7(1): 171). Therefore, NO signaling is a critical regulator of HSC behavior, influencing their migration, homing, proliferation, and differentiation.

Therapeutic strategies to restore physiological NO signaling include restoring NO production and bioavailability or by targeting its downstream signals. However while strategies have been proposed robust therapeutic successes are few, particularly with regard to effective conventional therapeutic small molecule drugs having the greatest potential for clinical development.

Thus remains a need in the art for reagents and methods to identify effective conventional therapeutic small molecules for drug development and drugs identified thereby for treating arterial endothelial cell and nitric oxide-related diseases and disorders.

### SUMMARY OF THE DISCLOSURE

Provided herein are reagents and methods for identifying effective conventional therapeutic small molecules for drug development and drugs identified thereby for treating arterial endothelial cell and nitric oxide-related diseases and disorders.

In particular embodiments, provided herein are reporter cell lines comprising arterial endothelial precursor cells genetically engineered with a recombinant expression construct wherein the cell is capable of identifying compounds that promote arterial endothelial cell differentiation, induce nitric oxide (NO) production, or both. In particular embodiments the reporter cell lines are H1 embryonic stem cells and alternatively the reporter cells lines are human induced pluripotent stem cells (iPSCs). Specifically the recombinant expression construct encodes NOS3-NLuc-2A-tdTomato.

Further provided herein are methods for identifying compounds capable of promoting arterial endothelial cell differentiation, inducing nitric oxide (NO) production, or both, in an arterial endothelial precursor cell, wherein the methods comprise contacting with a compound a reporter cell line genetically engineered with a recombinant expression construct wherein the cell is capable of identifying compounds that promote arterial endothelial cell differentiation, induce nitric oxide (NO) production, or both, wherein nitric oxide production, arterial endothelial cell differentiation, or both are detected when the cells are contacted with the compound.

Practice of the methods provided herein identify compounds capable of producing nitric oxide (NO) production, arterial endothelial cell (AEC) differentiation, or both. Particularly, such compounds include compounds identified as C21, C22, C23, C24, C25, and C33 for AEC differentiation, and C22, C23, C24, and C31 for inducing NO production.

Also provided herein are methods for inducing arterial endothelial cell differentiation in an arterial endothelial precursor that comprise contacting the precursor cell with a compound capable of inducing arterial endothelial cell differentiation. Specifically, the disclosed methods comprise an arterial endothelial precursor with a compound identified as C21, C22, C23, C24, C25, and C33.

Specifically provided herein are compounds capable of inducing NO production, when administered individually or in any advantageous combination, said compounds being identified by C22, C23, C24, and C31. Also provided are methods for identifying these and additional compounds having these capabilities when administered to a patient in need thereof.

Also specifically provided are methods for treating a disease or disorder associated with dysregulation of nitric oxide production in a cell, wherein the methods comprise administering to the patient in need thereof a therapeutically effective amount of a compound capable of producing nitric oxide production, arterial endothelial cell differentiation, or both. Specifically, the disclosed methods comprise administering to the patient a therapeutically effective amount of a compound identified as C22, C23, C24, or C31. Particular diseases or disorders are neurological diseases or disorders or cardiovascular diseases or disorders. Specific diseases and disorders include Parkinson's disease, Alzheimer's disease, amyotrophic lateral sclerosis, Huntington's disease, ischemic stroke, thrombosis, or atherosclerosis.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

The disclosure will be better understood and features, aspects, and advantages other than those expressly set forth above will become apparent when consideration is given to the following detailed description thereof. Such detailed description refers to the following drawings.

FIG. 1A is a diagram of CRISPR-mediated genetic modifications introducing the NOS3-NLuc-2A-tdTomato construct into the NOS3 gene locus in arterial endothelial precursor cells.

FIG. 1B is photograph of an ethidium bromide-stained agarose gel showing a unique fragment produced by PCR amplification of the junction produced by CRISPR-mediated introduction of the NOS3-NLuc-2A-tdTomato construct into the human NOS3 gene locus.

FIG. 1C is a bar graph of the copy number of NLuc and tdTomato in control cells without the construct wherein (−) is a negative control, (+) is a positive control having one copy of the construct, and (C4) is NOS3-NLuc-tdTomato clone 4. Due to the sensitivity of qPCR, copy number values between 0.6-1.4 are considered as single copy.

FIG. 1D illustrates a karyotype of arterial endothelial precursor cells showing normal chromosome number and structure after gene targeting.

FIG. 2A is a bar graph showing the number of “hits” from batched high throughput screening (HTS) of >20000 compounds. The x-axis is relative luciferase bioluminescent intensity normalized to the control (the average luminescent intensity of the whole plate).

FIG. 2B is a bar graph showing the number of “hits” from second batched screening of specific compounds. The x-axis is relative luciferase bioluminescent intensity normalized to the control (the average luminescent intensity of the whole plate).

FIG. 3A is a bar graph showing arterial endothelial cell differentiation in response to treatment on days 2 through 6 of different concentrations of compounds identified in compound screening assays. Differentiated arterial endothelial cells were identified by flow cytometry for CD144+ DLL4+ cells after 6 days of treatment. Dashed horizontal line shows the % level of the control at 3.3 μM (about 55%).

FIG. 3B is a bar graph showing arterial endothelial cell differentiation in response to three specific compounds at day 3-6 of differentiation.

FIG. 4A is a graph of nitric oxide (NO) production in response to contacting NOS3-NLuc-2A-tdTomato construct-containing reporter cells derived from arterial endothelial with compounds identified in the screen for 2 days. FIG. 4B shows mean fluorescence intensity (MFI) of DAF-FM from FIG. 4A.

FIG. 5 shows structures of screening compounds C21, C22, C23, C24, C25, and C31 from Table 2.

### DETAILED DESCRIPTION OF THE DISCLOSURE

Provided herein are reagents and methods for identifying effective conventional therapeutic small molecules for drug development and drugs identified thereby for treating arterial endothelial cell and nitric oxide-related diseases and disorders.

For the purposes of promoting an understanding of the principles of the disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other component, feature, element, or step or integer, step, or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by a person of ordinary skill in the art to which this disclosure belongs.

The term “contacting” includes the physical contact of at least one substance to another substance.

As used herein, “treatment” refers to the clinical intervention made in response to a disease, disorder, or physiological condition of the subject or to which a subject can be susceptible. The aim of treatment includes the alleviation or prevention of symptoms, slowing or stopping the progression or worsening of a disease, disorder, or condition and/or the remission of the disease, disorder, or condition.

The terms “effective amount” or “therapeutically effective amount” refer to an amount sufficient to effect beneficial or desirable biological and/or clinical results. In other words, a “therapeutically effective amount” is an amount that will provide some alleviation, mitigation, or decrease in at least one clinical symptom in the subject.

The terms “express” or “expression” refer to transcription and translation of a nucleic acid coding sequence resulting in production of the encoded polypeptide. “Express” or “expression” also refers to antigens that are expressed, inter alia, on cell surfaces.

As used herein, the term “subject” refers to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The human subject can be of any age (e.g., an infant, child, or adult).

The term “construct” refers to an artificially-designed segment of DNA that can be used to incorporate genetic material into a target cell (e.g., an hPSC).

The term “genetically engineered” as used herein refers to cells that have been manipulated using biotechnology to change the genetic makeup of the cells, including the transfer of genes within and across species boundaries to produce improved or non-naturally occurring cells. A human pluripotent stem cell or differentiated cell progeny thereof that contains an exogenous, recombinant, synthetic, and/or otherwise modified polynucleotide is considered to be a genetically engineered cell and, thus, non-naturally occurring relative to any naturally occurring counterpart. In some cases, genetically engineered cells contain one or more recombinant nucleic acids. In other cases, genetically engineered cells contain one or more synthetic or genetically engineered nucleic acids (e.g., a nucleic acid containing at least one artificially created insertion, deletion, inversion, or substitution relative to the sequence found in its naturally occurring counterpart). Procedures for producing genetically engineered cells are generally known in the art, for example, as described in Sambrook et al, Molecular Cloning, A Laboratory Manual (Fourth Edition), Cold Spring Harbor Press, Cold Spring Harbor, N.Y. (2012) and Doudna et al., CRISPR-Cas, A Laboratory Manual, Cold Spring Harbor Press, Cold Spring Harbor, N.Y. (2016).

The term “reporter” refers to a protein or a gene that encodes such a protein, whose expression correlates with expression of a second protein. The reporter gene is an exogenous coding region that is introduced into cells to provide means for measuring the promoter activity that controls the expression of the endogenous second protein. The reporter protein encoded by the reporter gene can be an enzyme that catalyzes a substrate to produce light (such as NanoLuc luciferase) or can be a fluorescent protein (such as tdTomato). Detecting expression of the reporter protein is therefore robust, sensitive, and rapid, and provides a mean to indirectly measure the expression of the endogenous second protein. Examples of reporter protein include, but are not limited to, GFP and its variants (YFP and CFP), RFP and its variants (mCherry and DsRed), alkaline phosphatase (AP), thymidine kinase (TK), Luciferase (such as NanoLuc, Firefly, renilla, and Gaussia), β-galactosidase, chloramphenicol acetyltransferase (CAT). The term “reporter cell line” refers to cell line that has incorporated the reporter gene into its genome and stably or transiently expresses the reporter gene.

A genetically engineered cell can be a cell that has been modified using a gene editing technique. Gene editing refers to a type of genetic engineering in which DNA is inserted, deleted, modified, or replaced in the genome of a living cell. In contrast to other genetic engineering techniques that can non-specifically or randomly insert genetic material into a host genome, gene editing can target the insertions to site-specific locations (e.g., AAVS1 alleles). Examples of gene editing techniques including, but are not limited to, restriction enzymes, zinc finger nucleases, TALENs, and CRISPR-Cas9.

A genetically engineered cell can be a stem cell (e.g., a human pluripotent stem cell) or any of their differentiated progeny cells (e.g., mesoderm cells, arterial endothelial precursor cells, arterial endothelial cells) that have been modified to express a gene or genes in said cell. Any of the cells described herein can be genetically engineered. In some embodiments, a genetically engineered cell refers to a cell that is differentiated from a cell that has been genetically engineered.

Human pluripotent stem cells (hPSCs), either embryonic or induced, provide access to the earliest stages of human development and offer a platform on which to derive a large number of progenitor cells or differentiated progeny cells thereof for inter alia cellular therapy and tissue engineering. Accordingly, the methods provided herein can comprise differentiating human pluripotent stem cells under conditions that promote differentiation of mesodermal cells to arterial endothelial precursor cells and differentiated products thereof.

As used herein, the term “mesoderm cell” refers to a cell having mesoderm-specific gene expression, capable of differentiating into a mesodermal lineage such as bone, muscle such as cardiac muscle, skeletal muscle, and smooth muscle (e.g., of the gut), connective tissue such as the dermis and cartilage, kidneys, the urogenital system, blood or hematopoietic cells, heart, and vasculature including endothelial precursor cells and particularly including arterial endothelial precursor cells and differentiated products thereof. Mesoderm-specific biomarkers include Brachyury (T). Culturing can take place on any appropriate surface (e.g., in two-dimensional or three-dimensional culture).

Human pluripotent stem cells (e.g., human ESCs or iPS cells) can be cultured in the absence of a feeder layer (e.g., a fibroblast feeder layer), a chemically defined medium, or a culture medium comprising poorly defined or undefined components. As used herein, “feeder-free” refers to culture conditions that are substantially free of a cell feeder layer. Cells grown under feeder-free conditions can be grown on a substrate, such as a chemically-defined substrate, and/or grown as an adherent culture. Suitable chemically-defined substrates include vitronectin.

As used herein, “pluripotent stem cells” appropriate for use according to a method of the invention are cells having the capacity to differentiate into cells of all three germ layers. Suitable pluripotent cells for use herein include human embryonic stem cells (hESCs) and human induced pluripotent stem (iPS) cells. As used herein, “embryonic stem cells” or “ESCs” mean a pluripotent cell or population of pluripotent cells derived from an inner cell mass of a blastocyst. See Thomson et al., Science 282:1145-1147 (1998). These cells can express Oct-4, SSEA-3, SSEA-4, TRA-1-60, and TRA-1-81. Pluripotent stem cells appear as compact colonies comprising cells having a high nucleus-to-cytoplasm ratio and prominent nucleolus. ESCs are commercially available from sources such as WiCell Research Institute (Madison, WI.).

As used herein, “induced pluripotent stem cells” or “iPS cells” refers to pluripotent cells or populations of pluripotent cells that can vary with respect to their differentiated somatic cell of origin, that can vary with respect to a specific set of potency-determining factors and that can vary with respect to culture conditions used to isolate them, but nonetheless are substantially genetically identical to their respective differentiated somatic cell of origin and display characteristics similar to higher potency cells, such as ESCs, as described herein. See, e.g., Yu et al., Science 318:1917-1920 (2007).

Induced pluripotent stem cells exhibit morphological properties (e.g., round shape, large nucleoli, and scant cytoplasm) and growth properties (e.g., doubling time of about seventeen to eighteen hours) akin to ESCs. In addition, iPS cells express pluripotent cell-specific markers (e.g., Oct-4, SSEA-3, SSEA-4, Tra-1-60, or Tra-1-81, but not SSEA-1). Induced pluripotent stem cells, however, are not immediately derived from embryos. As used herein, “not immediately derived from embryos” means that the starting cell type for producing iPS cells is a non-pluripotent cell, such as a multipotent cell or terminally differentiated cell, such as somatic cells obtained post-natally.

In some embodiments, any of the above-referenced cells are cultured in a xeno-free cell culture medium. Of importance for clinical therapies is the absence of xenogenic materials in the derived cell populations, i.e., no non-human cells, cell fragments, sera, proteins, and the like. Preferably, this invention arrives at xenogen-free differentiated cells by use of Collagen IV as a platform. In addition, the media disclosed herein are chemically defined and, in some embodiments, are made xeno-free, and incorporate human proteins, which can be produced using recombinant technology or derived from placenta or other human tissues in lieu of animal-derived proteins. In some embodiments, all proteins added to the medium are recombinant proteins.

As used herein, the terms “chemically defined medium” and “chemically defined culture medium” refer to a culture medium containing formulations of fully disclosed or identifiable ingredients, the precise quantities of which are known or identifiable and can be controlled individually. As such, a culture medium is not chemically-defined if (1) the chemical and structural identity of all medium ingredients is not known, (2) the medium contains unknown quantities of any ingredients, or (3) both. Standardizing culture conditions by using a chemically-defined culture medium minimizes the potential for lot-to-lot or batch-to-batch variations in materials to which the cells are exposed during cell culture. Accordingly, the effects of various differentiation factors are more predictable when added to cells and tissues cultured under chemically-defined conditions.

As used herein, the term “serum-free” refers to cell culture materials that do not contain serum or serum replacement, or that contains essentially no serum or serum replacement. For example, an essentially serum-free medium can contain less than about 1%, 0.9%, 0.8%, 0.7%, 0.6%, 0.5%, 0.4%, 0.3%, 0.2%, or 0.1% serum. “Serum free” also refers to culture components free of serum obtained from animal (e.g., fetal bovine) blood or animal-derived materials, which is important to reduce or eliminate the potential for cross-species viral or prion transmission. For avoidance of doubt, serum-containing medium is not chemically-defined.

Human pluripotent stem cells are genetically engineered for uses disclosed herein by CRIPSR-Cas9-mediated homologous recombination as described in more detail below. Generally, the CRISPR-Cas9 complex is produced using a sgRNA encoding sequence complementary to a genetic target in an arterial endothelial progenitor cell. In particular embodiments the target is a human gene, NOS3, which mediates NO production in these cells, For screening methods described herein, CRISPR-mediated genetic engineering is performed using a construct containing sequences comprising a recombinant expression construct encoding an expression cassette for a protein that produces a detectable product that can be used to monitor the expression of NOS3, flanked by sequences complementary to the genetic target in human pluripotent stem cells-derived arterial endothelial progenitor cells. In specific embodiments the target is the human NOS3 gene. Other genetic engineering methods have also been contemplated such as the FLP-FRT recombination system to introduce the recombinant gene into the cells (Shah et al., 2015, FEBS J. 282(17): 3323-3333).

Provided herein is a reporter cell line comprising an arterial endothelial precursor cell genetically engineered with a recombinant reporter construct, wherein the cell expresses a recombinant reporter capable of detecting compounds that promote arterial endothelial cell differentiation, induce nitric oxide synthase 3 (NOS3) expression, or both. The recombinant reporter construct is incorporated into the NOS3 gene locus and encodes a reporter protein including green fluorescent protein (GFP), yellow fluorescent protein (YFP), cyan fluorescent protein (CFP), red fluorescent protein (RFP), mCherry, DsRed, alkaline phosphatase (AP), thymidine kinase (TK), Luciferase, β-galactosidase, or chloramphenicol acetyltransferase (CAT). In some embodiments, the genetically engineered cells as described herein are useful for screening methods for detecting compounds that stimulate NO production in arterial endothelial cells. In some embodiments, the cells are genetically engineered to express a NOS3-NLuc-2A-tdTomato construct that is inserted in the human NOS3 genetic locus of the cells. Such constructs encode a NOS3 protein fused with NanoLuc reporter. As set forth more explicitly herein, these cells are cultured at useful quantities (e.g., 1 million cells) and screened for NOS3 expression as disclosed in the Examples below in the presence and absence of compounds tested for the capacity to induce NOS3 gene expression, as measured inter alia by the NanoLuc luminescence in the luciferase assay. Testing is advantageously performed using high throughput screening methods comprising 10,000-20,000 compounds, with more specific screening performed using compounds identified as having NOS3 expression-inducing characteristics in the high throughout screen. NOS3 catalyzes the formation of NO and endothelial cells are known to express NOS3 (and thus produce NO) more than PSCs (Zhang et al., 2017, Cell Reports 8(4): 907-918). Furthermore, NO signaling has been shown to be a critical regulator of HSC behavior, influencing their migration, homing, proliferation, and differentiation (Lapidot et al., 2020, Blood Sci. 2(2): 66-67; Hummar et al., 2020, Front Cell Dev Bio. 8: 610369; Jalnapurkar et al., 2016, Stem Cell Res Ther. 7(1): 171). Therefore, positive compounds are also tested for the capacity to induce NO production in AEC cells, as well as the capacity to induce AEC differentiation, after screening for positive NOS3 gene expression induction.

The compounds identified by the screening methods described herein are listed in Table 2. These compounds are selected from the indicated library but have not been described elsewhere to enhance NO production in AEC. Of these compounds C21, C22, C23, C24, and C31 increase NO production, and C21, C22, C23, C24, C25, C28, and C33 are also confirmed to increase AEC differentiation from PSCs.

Provided herein also is a method for treating a disease or disorder associated with dysregulation of nitric oxide production in a cell, comprising treating a patient in need thereof with a therapeutically effective amount of a compound that promotes NO production. Compounds that promote NO production are C21, C22, C23, C24, and C31. In some embodiments, the disease or disorder is a neurological disease or disorder or a cardiovascular disease or disorder. In some embodiments, the disease or disorder is Parkinson's disease, Alzheimer's disease, amyotrophic lateral sclerosis, Huntington's disease, ischemic stroke, thrombosis, or atherosclerosis.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The Examples set forth herein incorporate and rely on certain experimental and preparatory methods and techniques preformed as exemplified herein.

### Materials and Methods

### Construction of Vectors and CRISPR-Mediated Recombination.

The nucleotide sequence of the NOS3-NLuc-2A-tdTomato construct used in the experiments disclosed herein was constructed using conventional genetic engineering methods and has the following genetic topography as presented in Table 1 (wherein specific constructs can comprise components of the FLP-FRT recombination system at e.g. nt3086-3120 and nt4662-4696).

TABLE 1
Genetic Topography of NOS3-NLuc-2A-tdTomato Construct

| Nucleotide Sequence Number (nt) | Topography |
|---|---|
| 0-800 | NOS3 homology 5′ arm |
| 801-1310 | NanoLuc(NLuc) |
| 1311-1376 | 2A self-cleaving peptide sequence |
| 1401-2828 | tdTomato |
| 2984-3034 | SV40 poly A |
| 3132-3641 | PKG promoter sequence |
| 3721-4524 | Neomycin resistant gene |
| 4570-4646 | poly A |
| 4703-5519 | NOS3 homology 3′ arm |

In this construct, the NOS3 homology 5′ arm and NOS3 homology 3′ arm are sequences (SEQ ID NO: 2) having nucleic acid sequence homology to the human NOS3 gene wherein CRISPR-mediated homologous recombination was used to insert the construct specifically in the human NOS3 genetic locus. NanoLuc is a sequence encoding a 1.9 kDa luciferase enzyme using furimazine as a substrate and having a greater than 150-fold increase in bioluminescence (see, England et al., 2016, Bioconjugate Chemistry 27: 1175-1187), which permits detection of NOS3 activity in cells containing the vector. The neomycin resistance gene provides a selectable marker for producing useful quantities of the construct in bacteria and the promoter and polyA sequences enable expression of the construct in human cells. tdTomato is a fluorescence-producing protein that can be distinguished from the luciferase protein encoded by the NanoLuc sequence.

This construct was introduced into human pluripotent stem cells using CRISPR-mediated homologous recombination as follows. Human pluripotent stem cell (2×10⁶ cells) were resuspended in 500 μL E8 medium supplemented with 10 μM Y28732, 25 mM HEPE buffer, 10 μg of NOS3-NLuc-2A-Tom plasmid, 7.5 μg NOS3-gRNA (GGCGGCTCTCAGGGGCTGT, SEQ ID NO: 1) plasmid (as set forth in Hou et al., 2013, Efficient genome engineering in human pluripotent stem cells using Cas9 from Neisseria meningitidis, Proc. Natl. Acad. Sci. USA 110: 15664-15649), and 7.5 μg Cas9 plasmid (Addgene, Catalog #41815). Cells were transferred to a 4 mm cuvette (BioRad) and electroporation performed using a BioRad Gene Pulser Xcell Electroporation system at 250 V, 500 μF, and infinite resistance. Cells were then plated into appropriate Matrigel-coated culture dishes in E8 supplemented with 10 μM Y-27632. Cells were transferred to a 10-cm dish with E8+10 μM Y27632 after electroporation. Two to three days later, genecin was added and genecin-resistant clones isolated 7-10 days thereafter.

### Cell Culture and Arterial Endothelial Cell (AEC) Differentiation.

NOS3-NLuc-Tom human pluripotent stem cells (hPSCs), prepared as set forth above, were cultured in E8BAC medium for two days (day 0-2) to induce mesoderm formation. On days 2-5 cells received Five Factor medium daily, and AEC collected on day 6. More than 90% of the resulting cells were CD144⁺CXCR4⁺DLL4⁺, consistent with differentiation to AEC cells.

### Screening

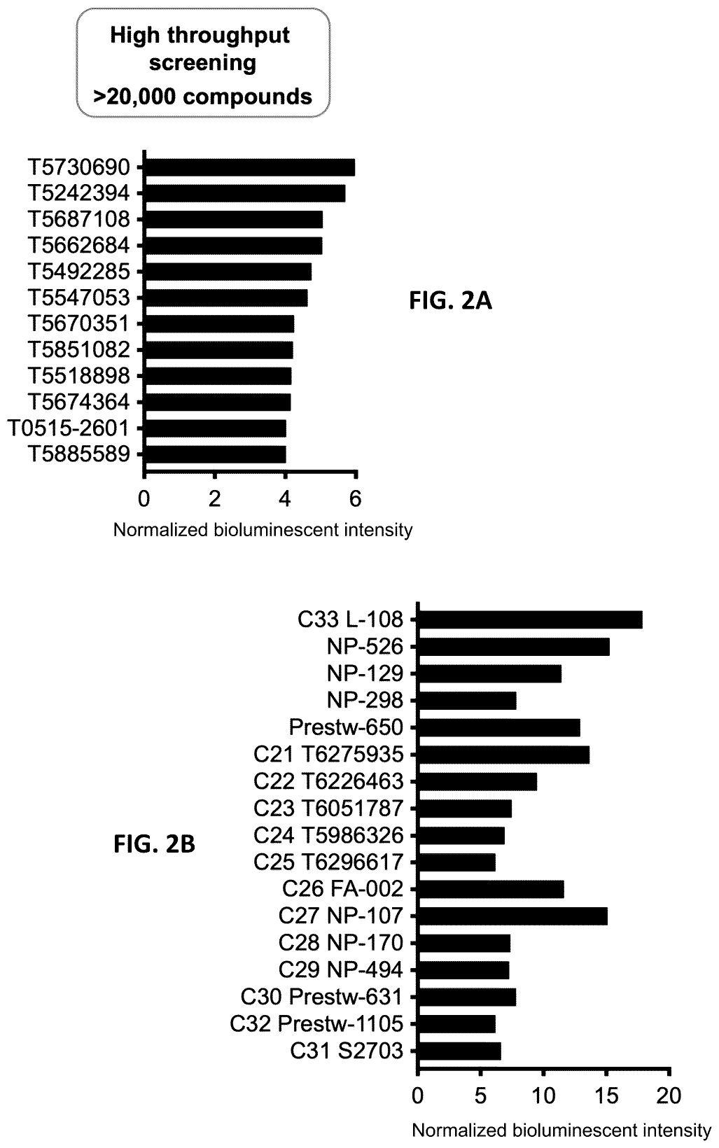

AECs (1×10⁶ cells/plate), prepared as set forth above, were seeded on a 384 well-plate with FVIR medium (the components thereof specified Table 3, below) for screening. Medium was changed every two days and compounds added to the medium on days 0, 2, and 4. On day 6 NOS3 expression was measured using a luciferase assay. Results of these experiments are shown in FIG. 2A and FIG. 2B. The hit compounds tested and the concentrations used are shown in Table 2.

TABLE 2
Hit Compounds Screened

| ID | Concentration | | Cat# | Compound name |
|---|---|---|---|---|
| C21 | 10 | μM | T6275935 | Z220377028¹ |
| C22 | 10 | μM | T6226463 | Z44288339¹ |
| C23 | 3 | μM | T6051787 | Z64462386¹ |
| C24 | 3 | μM | T5986326 | Z14203165¹ |
| C25 | 10 | μM | T6296617 | Z196164666¹ |
| C26 | 10 | μM | FA-002 | Docosahexaenoic acid (22:6 n-3)² |
| C27 | 10 | μM | NP-107 | Friedelin³ |
| C28 | 10 | μM | NP-170 | Leucomisine³ |
| C29 | 10 | μM | NP-494 | Hordenine sulfate³ |
| C30 | 10 | μM | Prestw-631 | Thiamine hydrochloride⁴ |
| C31 | 3 | μM | S2703 | GSK1838705A⁵ |
| C33 | 2 | μM | L-108 | 1-Octadecyl-2-methylglycero-3 |
| | | | | PC² |

Sources:
¹= Enamine 2011 Represntative Diversity Library;
²= ENZO Bioactive Lipid;
³= ENZO Natural Products Library;
⁴- Prestwick Chemical Library;
⁵= Sellack Kinase Inhibitors

### Nitric Oxide (NO) Production Assay

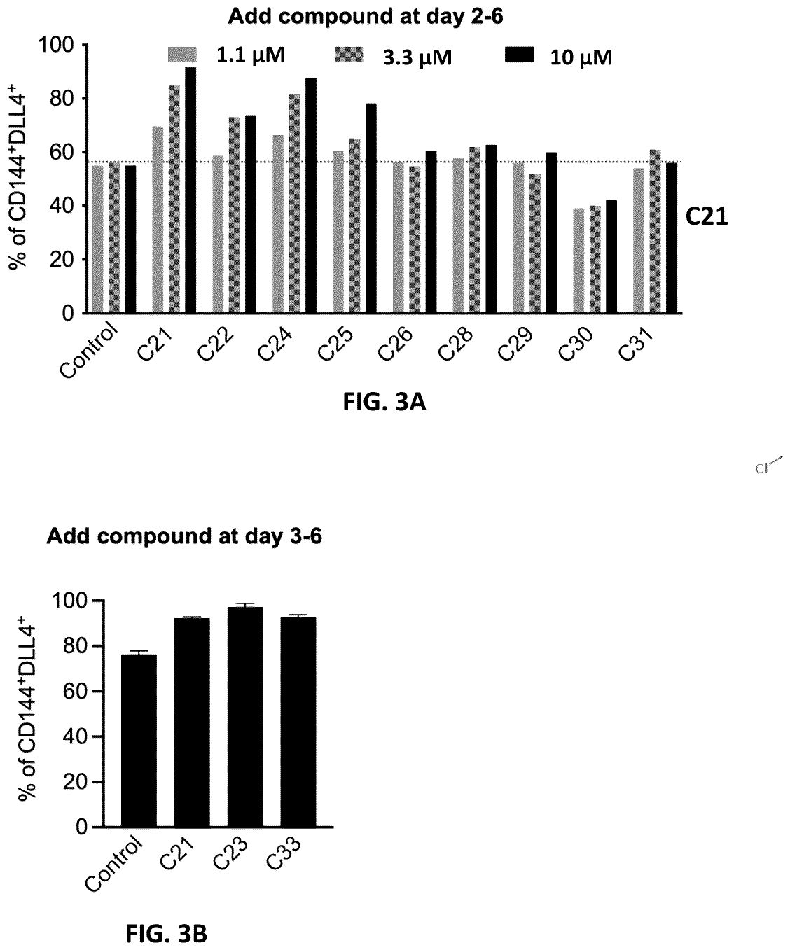

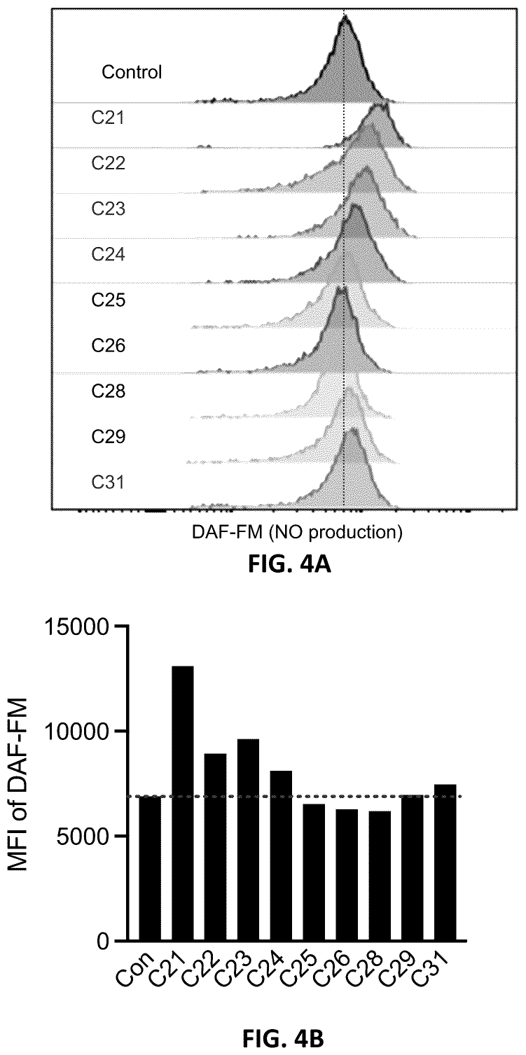

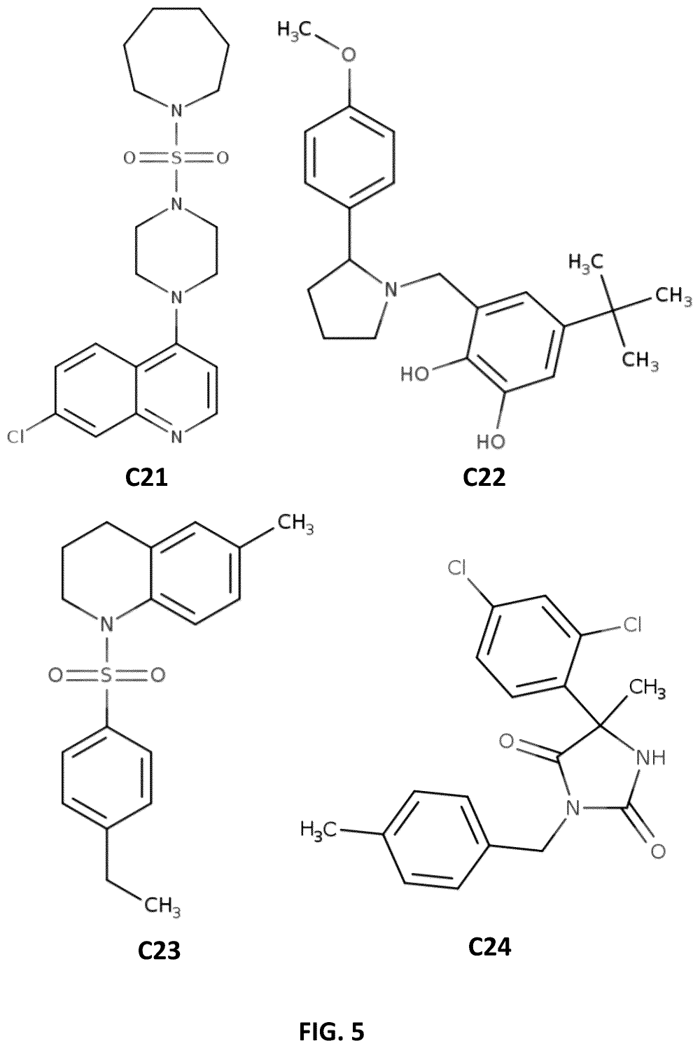

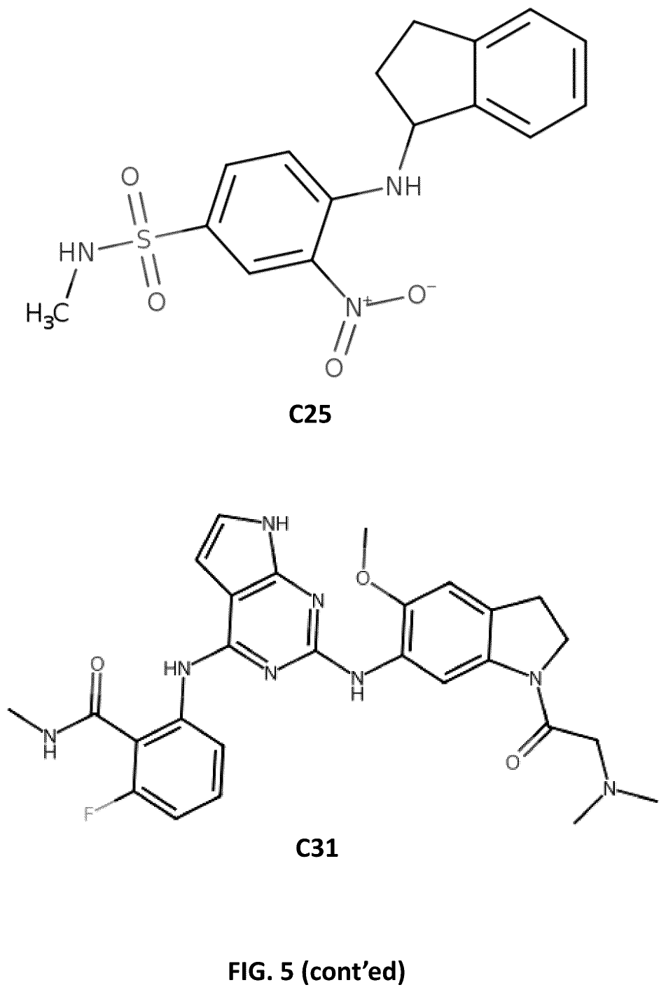

These AECs were seeded on a vitronectin-coated 24-well plate (1×10⁵ cells/well) with FVIR medium. Candidate compounds were added to the medium. Two days later, the medium was changed to fresh FVIR media containing 1 μM DAF-FM (4-Amino-5-Methylamino-2′,7′-Difluorofluorescein Diacetate). Cells were cultured for 30 mins and harvested for flow cytometric analysis. DAF-FM is nonfluorescent until it reacts with NO to form a fluorescent benzotriazole. Results of these experiments are shown in FIG. 3A, FIG. 3B, and FIG. 4.

TABLE 3
Medium Components

| Media components | E8 | E8BAC | E6 | Five factors/5F | FVIR |
|---|---|---|---|---|---|
| DF3S | + | + | + | + | + |
| Transferrin (10.7 μg/ml) | + | + | + | + | + |
| Insulin (20 μg/ml) | + | + | + | Insulin-free | + |
| FGF2 (100 ng/ml) | + | + | | + | + |
| TGFβ1 (2 ng/ml) | + | + | | | |
| BMP4 (5 ng/ml) | | + | | | |
| Activin A (25 ng/ml) | | + | | | |
| CHIR99021 (1 μM) | | + | | | |
| VEGFA165 (50 ng/ml) | | | | + | + |
| SB431542 (10 μM) | | | | + | + |
| RESV (5 μM) | | | | + | + |
| L690 (10 μM) | | | | + | |

### Example 1: Generation and Validation of Reporter Cell Line Expressing NOS3-NLuc-2A-tdTomato

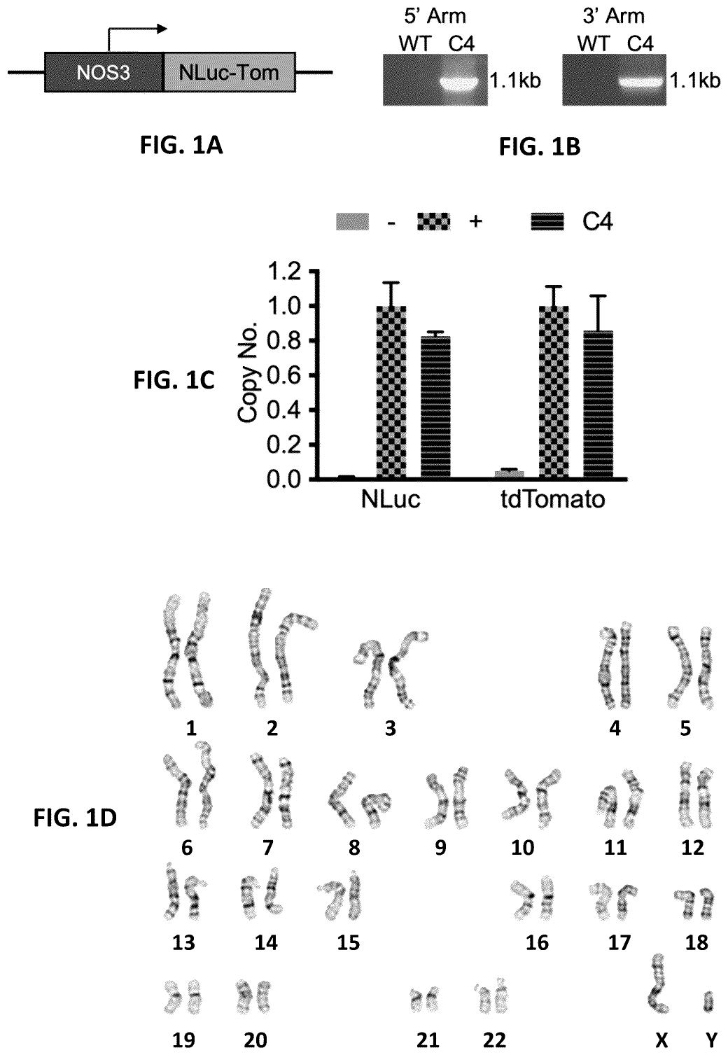

To generate a reporter PSC cell line, a donating plasmid NOS3-NLuc-2A-tdTomato as described in Table 1, containing a 5′ and 3′ end NOS3 homology sequence, and reporters NanoLuc and tdTomato sequences, was first generated using conventional methodologies known to those of ordinary skill in the art. The plasmid was then inserted into the NOS3 gene locus of the hPSCs by CRISPR-mediated homologous recombination. The hPSCs expressing NOS3-NLuc-2A-tdTomato were assessed for the insertion of the right constructs (FIG. 1B) and expression of the constructs (FIG. 1C). All NOS3-NLuc-2A-tdTomoato hPSCs had normal chromosome structures and numbers, indicating that the insertion did not cause any abnormal chromosomal constructs.

### Example 2: Use of NOS3-NLuc-2A-tdTomato hPSCs Cell Line for Screening Compounds that Promote NOS3 Expression

A high throughput screening of >20,000 compounds was carried out using the NOS3 reporter hPSCs cell lines. Briefly, the NOS3-NLuc-2A-tdTomato hPSCs were differentiated into AECs which were subsequently contacted with the tested compounds. Luciferase assay was performed and the level of fluorescence was measured. FIG. 2A and FIG. 2B show hit compounds in the first half of compounds and second half of compounds screening respectively. The top compounds C21-C33 were identified and summarized in Table 2. These compounds were selected from the indicated library and have not been shown to increase NOS3 expression previously. In summary, the results have shown that the reporter NOS3-NLuc-2A-tdTomato hPSCs cell line is a suitable model for high throughput screening and new compounds to promote NOS3 expression can be identified using this model.

### Example 3: Further Functional Studies to Confirm the Effects of the Hit Compounds

C21-C33 compounds were further tested to confirm their effects. Endothelial NOS3 is responsible for the generation of NO in the vascular endothelium and NO signaling has been shown to be a critical regulator of HSC behavior, influencing their migration, homing, proliferation, and differentiation (Lapidot et al., 2020, Blood Sci. 2(2): 66-67; Hummar et al., 2020, Front Cell Dev Bio. 8: 610369; Jalnapurkar et al., 2016, Stem Cell Res Ther. 7(1): 171). Thus, the effects of C21, C22, and C24-C31 on AEC differentiation from NOS3-NLuc-2A-tdTomato hPSCs were measured. Addition of these compounds, except C26-C30, from day 2 to day 6 of differentiation showed increase in percentage of AEC on day 6 in a dose-dependent manner, compared to control treatment (FIG. 3A). Addition of C21, C23, and C33 from day 3 to day 6 of differentiation also increased percentage of AEC on day 6 compared to control treatment (FIG. 3B). Furthermore, NO production assay with DAF-FM showed an increase in fluorescence indicating an increase in NO level with C22-24 and C31 compared to control treatment. In summary, the results showed that out of the 12 hit compounds (C21-C31 and C33) identified initially, 7 compounds (C21-C25, C31, and C33) were confirmed to have enhanced NO production and AEC differentiation.

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

SEQUENCE LISTING

SEQ ID NO: 1
Name: NOS3-gRNA
GGCGGCTCTCAGGGGCTGT
SEQ ID NO: 2
Name: NOS3-NLuc-2A-tdTomato
TGACAACCCCAAGGTGTGAGACCCTGAGGGCGCAATGGTAACCTGAAGATAGGGAG
AGAGGGGAGGACTCGCGCTCTCCAGCGGGGCACACCAACCACGGCCCTCCCGTGGC
CTCCCACGACCACTCAGCCACCCCTGCACACTCTGGCCCACCCTTGTGCCCCGGCCC
CTCTAGGCCCGCCTCCTCCCGCCCCTGCCCCGCCCCTTTGGCTCTGCCCCTGTTGACA
CCGCCCCAGGGCACGCAGGCCCCACCAGGCCCGCTCCGGAGACTTTCACGTCCAGG
GCCAGCCAGCAGCCCCGGGCTGCGCCCCCGCGCCCACCCCCACCAGGGCCCGCCCT
AACCCCGCCGCCCCGCAGACCTACGTGCAGGACATCCTGAGGACGGAGCTGGCTGC
GGAGGTGCACCGCGTGCTGTGCCTCGAGCGGGGCCACATGTTTGTCTGCGGCGATGT
TACCATGGCAACCAACGTCCTGCAGACCGTGCAGCGCATCCTGGCGACGGAGGGCG
ACATGGAGCTGGACGAGGCCGGCGACGTCATCGGCGTGCTGCGGGTGCGGAGGGGC
GGGCCGGGCCTGAGCGTGCGGGGTTCCTGCTAAGGTCTCCGAGTCGGGTTCTGATCC
ACTGTGCTCTTTTCCGACAGGATCAGCAACGCTACCACGAAGACATTTTCGGGCTCA
CGCTGCGCACCCAGGAGGTGACAAGCCGCATACGCACCCAGAGCTTTTCCTTGCAG
GAGCGTCAGTTGCGGGGCGCAGTGCCCTGGGCGTTCGACCCTCCCGGCTCAGACAC
CAACAGCCCCGTCTTCACACTCGAAGATTTCGTTGGGGACTGGCGACAGACAGCCG
GCTACAACCTGGACCAAGTCCTTGAACAGGGAGGTGTGTCCAGTTTGTTTCAGAATC
TCGGGGTGTCCGTAACTCCGATCCAAAGGATTGTCCTGAGCGGTGAAAATGGGCTG
AAGATCGACATCCATGTCATCATCCCGTATGAAGGTCTGAGCGGCGACCAAATGGG
CCAGATCGAAAAAATTTTTAAGGTGGTGTACCCTGTGGATGATCATCACTTTAAGGT
GATCCTGCACTATGGCACACTGGTAATCGACGGGGTTACGCCGAACATGATCGACTA
TTTCGGACGGCCGTATGAAGGCATCGCCGTGTTCGACGGCAAAAAGATCACTGTAA
CAGGGACCCTGTGGAACGGCAACAAAATTATCGACGAGCGCCTGATCAACCCCGAC
GGCTCCCTGCTGTTCCGAGTAACCATCAACGGAGTGACCGGCTGGCGGCTGTGCGA
ACGCATTCTGGCGGGAAGCGGAGCTACTAACTTCAGCCTGCTGAAGCAGGCTGGAG
ACGTGGAGGAGAACCCTGGACCTACCCGGGGGGTACCGGTCGCCACCATGGTGAGC
AAGGGCGAGGAGGTCATCAAAGAGTTCATGCGCTTCAAGGTGCGCATGGAGGGCTC
CATGAACGGCCACGAGTTCGAGATCGAGGGCGAGGGCGAGGGCCGCCCCTACGAG
GGCACCCAGACCGCCAAGCTGAAGGTGACCAAGGGCGGCCCCCTGCCCTTCGCCTG
GGACATCCTGTCCCCCCAGTTCATGTACGGCTCCAAGGCGTACGTGAAGCACCCCGC
CGACATCCCCGATTACAAGAAGCTGTCCTTCCCCGAGGGCTTCAAGTGGGAGCGCGT
GATGAACTTCGAGGACGGCGGTCTGGTGACCGTGACCCAGGACTCCTCCCTGCAGG
ACGGCACGCTGATCTACAAGGTGAAGATGCGCGGCACCAACTTCCCCCCCGACGGC
CCCGTAATGCAGAAGAAGACCATGGGCTGGGAGGCCTCCACCGAGCGCCTGTACCC
CCGCGACGGCGTGCTGAAGGGCGAGATCCACCAGGCCCTGAAGCTGAAGGACGGCG
GCCACTACCTGGTGGAGTTCAAGACCATCTACATGGCCAAGAAGCCCGTGCAACTG
CCCGGCTACTACTACGTGGACACCAAGCTGGACATCACCTCCCACAACGAGGACTA
CACCATCGTGGAACAGTACGAGCGCTCCGAGGGCCGCCACCACCTGTTCCTGGGGC
ATGGCACCGGCAGCACCGGCAGCGGCAGCTCCGGCACCGCCTCCTCCGAGGACAAC
AACATGGCCGTCATCAAAGAGTTCATGCGCTTCAAGGTGCGCATGGAGGGCTCCAT
GAACGGCCACGAGTTCGAGATCGAGGGCGAGGGCGAGGGCCGCCCCTACGAGGGC
ACCCAGACCGCCAAGCTGAAGGTGACCAAGGGCGGCCCCCTGCCCTTCGCCTGGGA
CATCCTGTCCCCCCAGTTCATGTACGGCTCCAAGGCGTACGTGAAGCACCCCGCCGA
CATCCCCGATTACAAGAAGCTGTCCTTCCCCGAGGGCTTCAAGTGGGAGCGCGTGAT
GAACTTCGAGGACGGCGGTCTGGTGACCGTGACCCAGGACTCCTCCCTGCAGGACG
GCACGCTGATCTACAAGGTGAAGATGCGCGGCACCAACTTCCCCCCCGACGGCCCC
GTAATGCAGAAGAAGACCATGGGCTGGGAGGCCTCCACCGAGCGCCTGTACCCCCG
CGACGGCGTGCTGAAGGGCGAGATCCACCAGGCCCTGAAGCTGAAGGACGGCGGCC
ACTACCTGGTGGAGTTCAAGACCATCTACATGGCCAAGAAGCCCGTGCAACTGCCC
GGCTACTACTACGTGGACACCAAGCTGGACATCACCTCCCACAACGAGGACTACAC
CATCGTGGAACAGTACGAGCGCTCCGAGGGCCGCCACCACCTGTTCCTGTACGGCAT
GGACGAGCTGTACAAGTAGGCGGCCGCGACTCTAGATCATAATCAGCCATACCACA
TTTGTAGAGGTTTTACTTGCTTTAAAAAACCTCCCACACCTCCCCCTGAACCTGAAAC
ATAAAATGAATGCAATTGTTGTTGTTAACTTGTTTATTGCAGCTTATAATGGTTACAA
ATAAAGCAATAGCATCACAAATTTCACAAATAAAGCATTTTTTTCACTGCATTCTAG
TTGTGGTTTGTCCAAACTCATCAATGTATCTTAAGGCGAGATCTTGAAGTTCCTATAC
TTTCTAGAGAATAGGAACTTCACGAAGTTATATTCTACCGGGTAGGGGAGGCGCTTT
TCCCAAGGCAGTCTGGAGCATGCGCTTTAGCAGCCCCGCTGGGCACTTGGCGCTACA
CAAGTGGCCTCTGGCCTCGCACACATTCCACATCCACCGGTAGGCGCCAACCGGCTC
CGTTCTTTGGTGGCCCCGTCGCGCCACCTTCTACTCCTCCCCTAGTCAGGAAGTTCCC
CCCCGCCCCGCAGCTCGCGTCGTGCAGGACGTGACAAATGGAAGTAGCACGTCTCA
CTAGTCTCGTGCAGATGGACAGCACCGCTGAGCAATGGAAGCGGGTAGGCCTTTGG
GGCAGCGGCCAATAGCAGCTTTGCTCCTTCGCTTTCTGGGCTCAGAGGCTGGGAAGG
GGTGGGTCCGGGGGCGGGCTCAGGGGGGGGCTCAGGGGCGGGGGGGGCGCCCGAA
GGTCCTCCGGAGGCCCGGCATTCTGCACGCTTCAAAAGCGCACGTCTGCCGCGCTGT
TCTCCTCTTCCTCATCTCCGGGCCTTTCGACCTGCAGCAGCACGTGTTGACAATTAAT
CATCGGCATAGTATATCGGCATAGTATAATACGACAAGGTGAGGAACTAAACCATG
GGATCGGCCATTGAACAAGATGGATTGCACGCAGGTTCTCCGGCCGCTTGGGTGGA
GAGGCTATTCGGCTATGACTGGGCACAACAGACGATCGGCTGCTCTGATGCCGCCGT
GTTCCGGCTGTCAGCGCAGGGGCGCCCGGTTCTTTTTGTCAAGACCGACCTGTCCGG
TGCCCTGAATGAACTGCAGGACGAGGCAGCGCGGCTATCGTGGCTGGCCACGACGG
GCGTTCCTTGCGCAGCTGTGCTCGACGTTGTCACTGAAGCGGGAAGGGACTGGCTGC
TATTGGGCGAAGTGCCGGGGCAGGATCTCCTGTCATCTCACCTTGCTCCTGCCGAGA
AAGTATCCATCATGGCTGATGCAATGCGGCGGCTGCATACGCTTGATCCGGCTACCT
GCCCATTCGACCACCAAGCGAAACATCGCATCGAGCGAGCACGTACTCGGATGGAA
GCCGGTCTTGTCGATCAGGATGATCTGGACGAAGAGCATCAGGGGCTCGCGCCAGC
CGAACTGTTCGCCAGGCTCAAGGCGCGCATGCCCGACGGCGAGGATCTCGTCGTGA
CCCATGGCGATGCCTGCTTGCCGAATATCATGGTGGAAAATGGCCGCTTTTCTGGAT
TCATCGACTGTGGCCGGCTGGGTGTGGCGGACCGCTATCAGGACATAGCGTTGGCTA
CCCGTGATATTGCTGAAGAGCTTGGCGGCGAATGGGCTGACCGCTTCCTCGTGCTTT
ACGGTATCGCCGCCCCCGATTCGCAGCGCATCGCCTTCTATCGCCTTCTTGACGAGT
TCTTCTGAGCGGGACTCTGGGGTTCGAATAAAGACCGACCAAGCGACGTCTGAGAG
CTCCCTGGCGAATTCGGTACCAATAAAAGAGCTTTATTTTCATGATCTGTGTGTTGGT
TTTTGTGTGCGGCGCGATAACTTCGTATAGCTGAAGTTCCTATACTTTCTAGAGAATA
GGAACTTCGCTAGCCTTTCCCTTCCAGTTCCGGGAGAGCGGCTGCCCGACTCAGGTC
CGCCCGACCAGGATCAGCCCCGCTCCTCCCCTCTTGAGGTGGTGCCTTCTCACATCT
GTCCAGAGGCTGCAAGGATTCAGCATTATTCCTCCAGGAAGGAGCAAAACGCCTCTT
TTCCCTCTCTAGGCCTGTTGCCTCGGGCCTGGGTCCGCCTTAATCTGGAAGGCCCCTC
CCAGCAGCGGTACCCCAGGGCCTACTGCCACCCGCTTCCTGTTTCTTAGTCGAATGT
TAGATTCCTCTTGCCTCTCTCAGGAGTATCTTACCTGTAAAGTCTAATCTCTAAATCA
AGTATTTATTATTGAAGATTTACCATAAGGGACTGTGCCAGATGTTAGGAGAACTAC
TAAAGTGCCTACCCCAGCTCATGTGGATTACAGTTTTTTTTTTTTGTTTTTTTTTTTTT
GAAACGGAGTCTCCCTCTGCCGCCCGGGCTGGAGTGCAGTGGCGTGATCTCAGCTCA
CTGCAACCTCCACCCCACAAGTTCAAGTGATTCTCCTGCCTCAGCCTCCCAAGTAGT
TGGGATTACAGGTGCCTGCCACCGCGCCCGGCTAGGTTTTGTATTTTTAGTAAAGAC
GGGGTTTCACCATCTTGGCCAGGCTGGTCTTGAACTCCTGACCTCGTGATCCAACCG
CCTCAGCCTCCCAAAGTGCTGGGATTACAGGTGTGAGCTACTGCACCCGGCGTGGAT
TACAATTATAAAATGACAAGATTTCTGTTTTAACCTGTGCAGTTGTGGGTATGTGGT
GGGGAAAGGGGTCATTCTTTTGACAGAGT

1. A reporter cell line comprising an arterial endothelial precursor cell genetically engineered with a recombinant reporter construct, wherein the cell expresses a recombinant reporter capable of detecting compounds that promote arterial endothelial cell differentiation, induce nitric oxide synthase 3 (NOS3) expression, or both.

2. The reporter cell line of claim 1 comprising H1 embryonic stem cells.

3. The reporter cell line of claim 1 comprising human induced pluripotent stem cells (iPSC).

4. The reporter cell line of claim 1, wherein the recombinant reporter construct is incorporated into a NOS3 gene locus in the reporter cell line's genome.

5. The reporter cell line of claim 1, wherein the recombinant reporter construct encodes a reporter protein that is green fluorescent protein (GFP), yellow fluorescent protein (YFP), cyan fluorescent protein (CFP), red fluorescent protein (RFP), mCherry, DsRed, alkaline phosphatase (AP), thymidine kinase (TK), Luciferase, β-galactosidase, or chloramphenicol acetyltransferase (CAT).

6. A recombinant expression construct comprising a nucleotide sequence that is 90% identical to the nucleotide sequence of SEQ ID NO: 2.

7. A method for identifying compounds capable of promoting arterial endothelial cell differentiation, inducing nitric oxide (NO) production, or both, in the arterial endothelial precursor cell of claim 1, comprising contacting the arterial endothelial precursor cell with the compound and detecting nitric oxide production, arterial endothelial cell differentiation, or both.

8. A compound identified by the method of claim 7.

9. The compound of claim 8, wherein the compound is C21, C22, C23, C24, C25, C28, C31, or C33.

10. A method of inducing arterial endothelial cell differentiation in an arterial endothelial precursor cell comprising contacting the precursor cell with a compound identified according to claim 8.

11. The method of claim 10 wherein the compound is C21, C22, C23, C24, C25, C28, or C33.

12. A method of inducing nitric oxide production in a cell comprising contacting the cell with a compound identified according to claim 8.

13. The method of claim 12 wherein the compound is C21, C22, C23, C24, or C31.

14. A method for treating a disease or disorder associated with dysregulation of nitric oxide production in a cell, comprising treating a patient in need thereof with a therapeutically effective amount of a compound of claim 8.

15. The method of claim 14 wherein the disease or disorder is a neurological disease or disorder or a cardiovascular disease or disorder.

16. The method of claim 15, wherein the disease or disorder is Parkinson's disease, Alzheimer's disease, amyotrophic lateral sclerosis, or Huntington's disease.

17. The method of claim 15, wherein the disease or disorder is ischemic stroke, thrombosis, or atherosclerosis.

18. The method of claims 14-17, wherein the compound is C21, C22, C23, C24, or C31.
